(19) United States
(12) Patent Application Publication
(10) Pub. No.: US 2008/0312220 A1
(43) Pub. Date: Dec. 18, 2008

(54) Oxadiazole Derivatives with Crth2 Receptor Activity

(76) Inventors: Jean-Marie Receveur, Hoersholm (DK); Ann Christensen, Hoersholm (DK); Marie Grimstrup, Hoersholm (DK); Thomas Hoegberg, Hoersholm (DK)

Correspondence Address: BANNER & WITCOFF, LTD., 1100 13th STREET, N.W., SUITE 1200, WASHINGTON, DC, 20005-4051, US

(21) Appl. No.: 12/094,907

(22) PCT Filed: Nov. 22, 2006
(86) PCT No.: PCT/EP2006/011216

§ 371 Date: May 23, 2008

(30) Foreign Application Priority Data
Nov. 30, 2005 (GB) 0524428.0

(51) Int. Cl. A61K 31/541; C07D 271/06; A61K 31/4245; A61K 31/454; C07D 417/12; C07D 413/12; A61K 31/5377

(52) U.S. Cl. 514/227.8; 548/131; 514/364; 514/326; 546/209; 544/138; 514/236.2; 544/58.2

(57) ABSTRACT

Compounds of formula are CRTH2 ligands, useful for treatment of inflammatory, autoimmune, respiratory or allergy disease: wherein R₁ is hydrogen or methyl and R₂ is optionally substituted cycloalkyl, or optionally substituted non-aromatic heterocyclyl having to 6 ring atoms; or R₁ and R₂, taken together with the carbon atom to which they are attached form an optionally substituted cycloalkyl, or optionally substituted non-aromatic heterocyclyl having 4 to 6 ring atoms: or R₁ and R₂, taken together with the carbon atom to which they are attached form an optionally substituted cycloalkyl, or optionally substituted cycloalkyl, or optionally substituted non-aromatic heterocyclyl ring having 4 to 6 ring atoms; R is hydrogen or an optional substitutent by 1, 2 or 3 optional substituents; A is hydrogen or C₁-C₃ alkyl; and ring Ar is an optionally substituted phenyl or 5- or 6-membered monocyclic heteroaryl ring.

This invention relates to the use of a class of compounds which are ligands of the CRTH2 receptor (Chemoattractant Receptor-homologous molecule expressed on T Helper cells type 2), in the treatment of diseases responsive to modulation of CRTH2 receptor activity, principally diseases having a significant inflammatory component. The invention also relates to novel members of that class of ligands and pharmaceutical compositions containing them.

Many classes of antiinflammatory agents are known, including the non-steroidal antiinflammatory compounds known as NSAIDs and the inhibitors of cyclooxygenase (COX-1 and COX-2). Benzoylphenylacetic acid and some benzophenone derivatives with carboxymethoxy substituents in one of the rings have been identified as antiinflammatory agents (see, for example, Khanum et. al. Bioorganic Chemistry Vol 32, No. 4, 2004, pages 211-222 and the references cited therein). Some o-phenyl carbamoyl-phenoxyacetic acids and o-benzamido-phenoxymethyl tetrazoles have been reported as potential antiinflammatory agent, see for example Drain et. al. J. Pharm. Pharmac., 1971, 23, 857-864, and ibid 1970, 22, 684-693. WO 99/15520 discloses a few benzophenone derivatives with carboxymethoxy or tetrazolylmethoxy substituents in one of the rings, synthesised as members of a group of compounds said to have activity as inhibitors of peroxisome proliferator-activated receptor (PPAR), and utility in a variety of disease states including diabetes, cardiac disease, and circulatory disease.

The natural ligand of the G-protein coupled receptor CRTH2 is prostaglandin D2. As its name implies, CRTH2 is expressed on T helper cells type 2 (Th2 cells) but it is also known to be expressed on eosinophils and basophil cells. Cell activation as a result of binding of PGD2 to the CRTH2 receptor results in a complex biological response, including release of inflammatory mediators. Elevated levels of PGD2 are therefore associated with many diseases which have a strong inflammatory component, such as asthma, rhinitis and allergies. Blocking binding of PGD2 to the CRTH2 receptor is therefore a useful therapeutic strategy for treatment of such diseases.

Some small molecule ligands of CRTH2, apparently acting as antagonists of PGD2, are known, for example as proposed in the following patent publications: WO 03/097042, WO 03/097598, WO 03/066046, WO 03/066047, WO 03/101961, WO 03/101981, GB 2388540, WO 04/089885 and WO 05/018529.

The structures of PGD2 antagonist compounds referred to in some of the foregoing publications have a bicyclic or tricyclic core ring system related to the indole core of indomethacin, a known anti-inflammatory agent, now known to bind to CRTH2. The present invention arises from the identification of a class of compounds having a monocyclic core whose substituent moieties are selected and orientated by the monocyclic core to interact with and bind to CRTH2. The class of compounds with which this invention is concerned are thus capable of modulating CRTH2 activity, and are useful in the treatment of diseases which benefit from such modulation, for example asthma, allergy and rhinitis.

Our copending international application PCT/EP2005/005884 is concerned with the use of a compound of formula (IA) or a salt, hydrate or solvate thereof in the manufacture of a composition for the treatment of disease responsive to modulation of CRTH2 receptor activity:

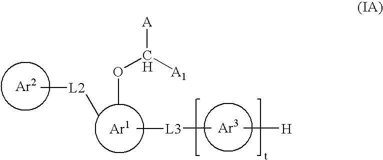

wherein
A represents a carboxyl group —COOH, or a carboxyl bioisostere;
A₁ is hydrogen or methyl;
ring Ar¹ is an optionally substituted phenyl ring or 5- or 6-membered monocyclic heteroaryl ring, in which AA₁CHO— and L2 are linked to adjacent ring atoms;
rings Ar², Ar³ each independently represent a phenyl or 5- or 6-membered monocyclic heteroaryl ring, or a bicyclic ring system consisting of a 5- or 6-membered carbocyclic or heterocyclic ring which is benz-fused or fused to a 5- or 6-membered monocyclic heteroaryl ring, said ring or ring system being optionally substituted;
t is 0 or 1;
L2 and L3 each independently represents a divalent radical of formula -(Alk¹)ₘ-(Z)ₙ-(Alk²)ₚ wherein
m, n and p are independently 0 or 1,
Alk¹ and Alk² are independently optionally substituted straight or branched chain C₁-C₃ alkylene or C₂-C₃ alkenylene radicals which may contain a compatible —O—, —S— or —NR— link wherein R is hydrogen or C₁-C₃ alkyl, and
Z is —O—; —S—; —C(═O)—; —SO₂—; —SO—; —NR—, —NRSO₂—, —SO₂NR—, —C(═O)NR—, —NRC(═O)—, —NRCONH—, —NHCONR—, —NRC(═NR)NH—, —NHC(═NR)NR—, —C(R)═N—NR—, or —NR—N═C(R)— wherein R is hydrogen or C₁-C₃ alkyl; or a divalent 5- or 6-membered monocyclic carbocyclic or heterocyclic radical,
   Provided that
(A) the total length of L2 and L3 does not exceed that of an unbranched saturated chain of 10 carbon atoms; and
(B) L2 is not —C(═O)—, —C(═O)NR—, or —NRC(═O)— when Ar² is optionally substituted phenyl; and
(C) (a) L2 is not a bond and (b) p in L2 is not 0 when n is 1 and Z is aryl or heteroaryl, and
(D) (a) L2 is not —O—, —SO₂—, —NR—, —CHR^{X}R^{Y}— or —CH(R^{X})(OR^{Y})—, wherein R^{X} and R^{Y} are independently hydrogen, halogen, C₁-C₆ alkyl, C₂-C₆ alkenyl, C₂-C₆ alkynyl, or C₃-C₇ cycloalkyl, or join to form a ring, and (b) when p is 1 and n is 1 and Z is aryl or heteroaryl then Alk² is not —CHR^{X}R^{Y}— or —CH(R^{X})(OR^{Y})—, wherein R^{X} and R^{Y} are independently hydrogen, halogen, C₁-C₆ alkyl, C₂-C₆ alkenyl, C₂-C₆ alkynyl, or C₃-C₇ cycloalkyl, or join to form a ring.

Since it is not yet published, a copy of the description part of the above-mentioned document PCT/EP2005/005884 is attached as an APPENDIX.

The present invention is concerned with a compounds related to those of PCT/EP2005/005884, having an arylmethyloxadiazolyl radical attached to a phenyl ring corresponding to ring Ar¹ of the compounds of PCT/EP2005/005884, said radical having a specific substitution as described below, on the carbon atom between the aryl and oxadiazole rings.

According to the present invention, there is provided compound of formula (I) or a salt, hydrate or solvate thereof other than {4-bromo-2-[3-(1-phenylcyclopropyl)-[1,2,4]oxadiazol-5-yl]phenoxy}acetic acid or a salt, hydrate or solvate thereof:

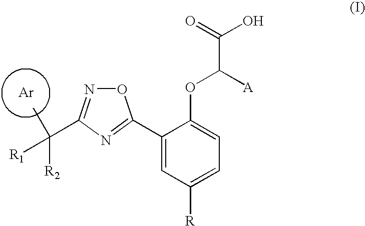

wherein
R₁ is hydrogen or methyl and R₂ is optionally substituted cycloalkyl, or optionally substituted non-aromatic heterocyclyl having 4 to 6 ring atoms; or R₁ and R₂, taken together with the carbon atom to which they are attached form an optionally substituted cycloalkyl, or optionally substituted non-aromatic heterocyclyl ring having 4 to 6 ring atoms;
R is hydrogen or an optional substituent;
the phenyl ring containing the substituent R is optionally substituted by 1, 2 or 3 optional substituents;
A is hydrogen or C₁-C₃ alkyl;
ring Ar is an optionally substituted phenyl or 5- or 6-membered monocyclic heteroaryl ring.

The compound {4-bromo-2-[3-(1-phenylcyclopropyl)-[1,2,4]oxadiazol-5-yl]phenoxy}-acetic acid (and its salts, hydrates and solvates) has formula (I) above wherein A is hydrogen, Ar is phenyl, R is bromo, and R₁ and R₂ taken together with the carbon atom to which they are attached form a cyclopropyl ring. That compound is excluded from all aspects of the present invention because it is specifically disclosed in PCT/EP2005/005884.

In another aspect, the invention provides a method of treatment of a subject suffering from a disease responsive to modulation of CRTH2 receptor activity, which comprised administering to the subject an amount of a compound (1) as defined and described above effective to ameliorate the disease.

In particular, compounds with which the invention is concerned are useful in the treatment of disease associated with elevated levels of prostaglandin D2 (PGD2) or one or more active metabolites thereof.

Examples of such diseases include asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behçet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

However, the compounds with which the invention is concerned are primarily of value for the treatment asthma, rhinitis, allergic airway syndrome, and allergic rhinobronchitis.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyridazinyl, triazinyl, indolyl and indazolyl.

As used herein the unqualified term “heterocyclyl” or “heterocyclic” includes “heteroaryl” as defined above, and in addition means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, cycloalkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo, phenyl, phenoxy, —COOR^{A}, —COR^{A}, —OCOR^{A}, —SO₂R^{A}, —CONR^{A}R^{B}, —SO₂NR^{A}R^{B}, —NR^{A}R^{B}, OCONR^{A}R^{B}, —NR^{B}COR^{A}, —NR^{B}COOR^{A}, —NR^{B}SO₂ORA or —NR^{A}CONR^{A}R^{B} wherein R^{A} and R^{B} are independently hydrogen or a (C₁-C₆)alkyl group or, in the case where R^{A} and R^{B} are linked to the same N atom, R^{A} and R^{B} taken together with that nitrogen may form a cyclic amino ring. Where the substituent is phenyl or phenoxy, the phenyl ring thereof may itself be substituted by any of the above substituents except phenyl or phenoxy. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as ammonium hydroxide; alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (1) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

Use of prodrugs, such as esters, of compounds (1) with which the invention is concerned is also part of the invention.

For use in accordance with the above aspects of the invention the following structural characteristics may be present, in any compatible combination, in the compounds (1):
In the case where R₁ is hydrogen or methyl, and R₂ is optionally substituted cycloalkyl, R₂ may be, for example, optionally substituted cyclopropyl, cyclobutyl, cyclopentyl, or cyclohexyl;
In the case where R₁ is hydrogen or methyl, and R₂ is optionally substituted non-aromatic heterocyclyl having 4 to 6 ring atoms, the R₂ ring radical may be, for example, of formula

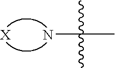

wherein the ring contains 4 to 6 ring atoms, and X is selected from —CH₂—, —CH(C₁-C₃alkyl)-, —C(C₁-C₃alkyl)₂-, —CH(cycloalkyl), —CH(NH₂)—, —C(CH₃)(NH₂)—, —CH(NH(C₁-C₃alkyl))-, —CH(N(C₁-C₃alkyl)₂)—, —CH(NH(cycloalkyl))-, —CH(NHCO(C₁-C₃alkyl))-, —CH(NHCO(cycloalkyl))-, —CH(NHSO₂(C₁-C₃alkyl))-, —CH(NHSO₂(cycloalkyl))-, —CH(OH)—, —CH(C₁-C₃alkoxy)-, —CH(cycloalkyloxy)-, —CO—, —SO₂—, —O—, —NH—, —N(C₁-C₃alkyl)-, —N(cycloalkyl)-, —CONH—, —CON(C₁-C₃alkyl)-, —CON(cycloalkyl)-, —N(CO(OC₁-C₃alkyl))-, —N(CO(O-cycloalkyl))-, —N(CO(CH₂OH))—, —SO₂NH—, —SO₂N(C₁-C₆alkyl)-, —SO₂N(cycloalkyl)-, —N(SO₂(C₁-C₃alkyl))-, —N(SO₂(cycloalkyl))-, —N(CO(C₁-C₃alkyl))-, or —N(CO(cycloalkyl))-. Of the foregoing options for X, the following are presently preferred: —CH₂—, —SO₂—, —CO—, —O—, —N(SO₂(C₁-C₃alkyl))-, —N(SO₂(cycloalkyl))-, —N(CO(C₁-C₃alkyl))-, —N(CO(cycloalkyl))-, —CONH—, —CON(C₁-C₃alkyl)-, and —CON(cycloalkyl)-. Where, in the foregoing options for X, a C₁-C₃alkyl group is present, methyl is often preferred, and where a cycloalkyl group is present, cyclopropyl is often preferred.
In compounds of the invention, when R₁ is hydrogen or methyl, specific examples of groups R₂ include those present in the compounds of the examples herein.
In the case where R₁ and R₂, taken together with the carbon atom to which they are attached form an optionally substituted cycloalkyl ring, that ring may be, for example, optionally substituted cyclopropyl, cyclobutyl, cyclopentyl, or cyclohexyl.
In the case where R₁ and R₂, taken together with the carbon atom to which they are attached form an optionally substituted non-aromatic heterocyclyl ring having 4 to 6 ring atoms, that ring may be, for example, of formula

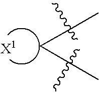

wherein the ring contains 4 to 6 ring atoms and X¹ is selected from —CH₂—, —CH(C₁-C₃alkyl)-, —C(C₁-C₃alky)₂)—, —CH(cycloalkyl), —CH(NH₂)—, —CMe(NH₂)—, —CH(NH(C₁-C₃alkyl))-, —CH(N(C₁-C₃alkyl)₂)—, —CH(NH(cycloalkyl))-, —CH(NHCO(C₁-C₃alkyl))-, —CH(NHCO(cycloalkyl))-, —CH(NHSO₂(C₁-C₃alkyl))-, —CH(NHSO₂(cycloalkyl))-, —CH(OH)—, —CH(C₁-C₃alkoxy)-, —CH(cycloalkyloxy))-, —SO₂—, —O—, —NH—, —N(C₁-C₃alkyl)-, —N(cycloalkyl)-, —CONH—, —CON(C₁-C₃alkyl)-, —CON(cycloalkyl)-, —SO₂NH—, —SO₂N(C₁-C₆alkyl)-, —SO₂N(cycloalkyl)-, —N(SO₂(C₁-C₃alkyl))-, —N(SO₂(cycloalkyl))-, —N(CO(OC₁-C₃alkyl))-, —N(CO(O-cycloalkyl))-, —N(CO(CH₂OH))—, —N(CO(C₁-C₃alkyl))-, or —N(CO(cycloalkyl))-. Of the foregoing options for X¹, the following are presently preferred: —CH₂—, —SO₂—, —O—, —CONH—, —CON(C₁-C₃alkyl)-, —CON(cycloalkyl)-, —N(SO₂(C₁-C₃alkyl))-, —N(SO₂(cycloalkyl))-, —N(CO(C₁-C₃alkyl))-, and —N(CO(cycloalkyl))-. Where, in the foregoing options for X¹, a C₁-C₃alkyl group is present, methyl is often preferred, and where a cycloalkyl group is present, cyclopropyl is often preferred
In compounds of the invention, when R₁ and R₂, taken together with the carbon atom to which they are attached form a ring, specific examples such rings include those present in the compounds of the examples herein.
A may be hydrogen, methyl, ethyl, n- or iso-propyl. Presently preferred are compounds wherein A is hydrogen or methyl.
R may be, for example, a substituent selected from fluoro, chloro, bromo, (C₁-C₃)alkyl, cycloalkyl, trifluoromethyl, (C₁-C₃)alkoxy, (C₁-C₃)alkylmercapto, trifluoromethoxy, trifluoromethylthio, cyano, (C₁-C₃alkyl)SO₂—, NH₂SO₂—, (C₁-C₃alkyl)NHSO₂—, (C₁-C₃alkyl)₂NSO₂—, (cycloalkyl)NHSO₂—, NH₂CO—, (C₁-C₃alkyl)NHCO—, (C₁-C₃alkyl)₂NHCO—, and (cycloalkyl)NHCO—.
Ring Ar corresponds to the ring Ar² of the compounds of APPENDIX 1, and may be, for example, any of the Ar² groups mentioned therein or present in the exemplified compounds of APPENDIX 1. Thus, the present Ar group may be, for example, optionally substituted phenyl, pyridyl, pyrimidyl, diazolyl, oxazolyl, triazinyl, quinolinyl, pyrrollyl, furanyl, or thiazolyl.
Likewise, optional substituents in Ar may be, for example, any of the optional Ar² substituents mentioned in, or present in the exemplified compounds of, APPENDIX 1. Thus, optional substituents in the present ring Ar may be selected from fluoro, chloro, bromo, (C₁-C₃)alkyl, trifluoromethyl, (C₁-C₃)alkoxy, trifluoromethoxy, trifluoromethylthio, dimethylamino, cyano, (C₁-C₃alkyl)SO₂—, NH₂SO₂—, (C₁-C₃alkyl)NHSO₂—, and (C₁-C₃alkyl)₂NSO₂—,
The phenyl ring containing R in the present compounds of formula (I) corresponds to the ring Ar¹ of the compounds of APPENDIX 1, so any of the optional Ar¹ substituents mentioned in, or present in the exemplified compounds of, APPENDIX 1 may also be present in the R-containing phenyl ring of the compounds of this invention, Thus, such optional substituents may be selected from fluoro, chloro, bromo, cyano, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, NH₂CO—, (C₁-C₃alkyl)NHCO—, (C₁-C₃alkyl)₂NHCO—, (cycloalkyl)NHCO—, C₁-C₆alkyl, C₁-C₆alkoxy, cycloalkyl, aryl, aryloxy, aryl(C₁-C₆) or aryl(C₁-C₆ alkoxy)-.

The invention also includes pharmaceutical compositions comprising a compound belonging to the above described compounds of formula (I) together with a pharmaceutically acceptable carrier.

### Compositions

As mentioned above, the compounds with which the invention is concerned are capable of modulating CRTH2 activity, and are useful in the treatment of diseases which benefit from such modulation. Examples of such diseases are referred to above, and include asthma, allergy and rhinitis.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The drug may also be formulated for inhalation, for example as a nasal spray, or dry powder or aerosol inhalers.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds with which the invention is concerned may be administered alone, or as part of a combination therapy with other drugs used for treatment of diseases with a major inflammatory component. In the case of asthma, rhinitis, and allergic airway syndrome such drugs include corticosteroids, long-acting inhaled beta agonists, cromolyn, nedocromil, theophylline, leukotriene receptor antagonists, antihistamines, and anticholinergics (e.g. ipratropium), and are often administered as nasal sprays, dry powder or aerosol inhalers.

In the case of arthritis and related inflammatory diseases other known drugs include glucocorticoids, NSAIDs (Non Steroidal Anti-inflammatory Drugs—conventional prostaglandin synthesis inhibitors, COX-2 inhibitors, salicylates), and DMARDs (disease-modifying anti-rheumatic drugs such as methotrexate, sulfasalazine, gold, cyclosporine).

### Synthetic Routes

There are multiple synthetic strategies for the synthesis of the compounds (1) with which the present invention is concerned, but all rely on known chemistry, known to the synthetic organic chemist. Thus, compounds according to Formula I can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”.

In particular, the compounds of the present invention may be synthesised by the method described in the general routes summarised in the following three paragraphs, and in the Examples below, or by methods generally described in relation to the compounds of APPENDIX 1.

The core oxadiazole of Formula I is normally formed by condensation of amidoximes and optionally substituted methyl benzoates. The acidic side chains are introduced by base catalysed substitution of the phenol with e.g. bromoacetate or 2-bromoproprionate esters followed by alkaline hydrolysis of the ester. The R1 and R2 groups are either available in commercial starting materials or introduced at the nitrile stage utilising base catalysed nucleophilic substitutions or R₁ and R₂ are introduced after assembly of the oxadiazole system.

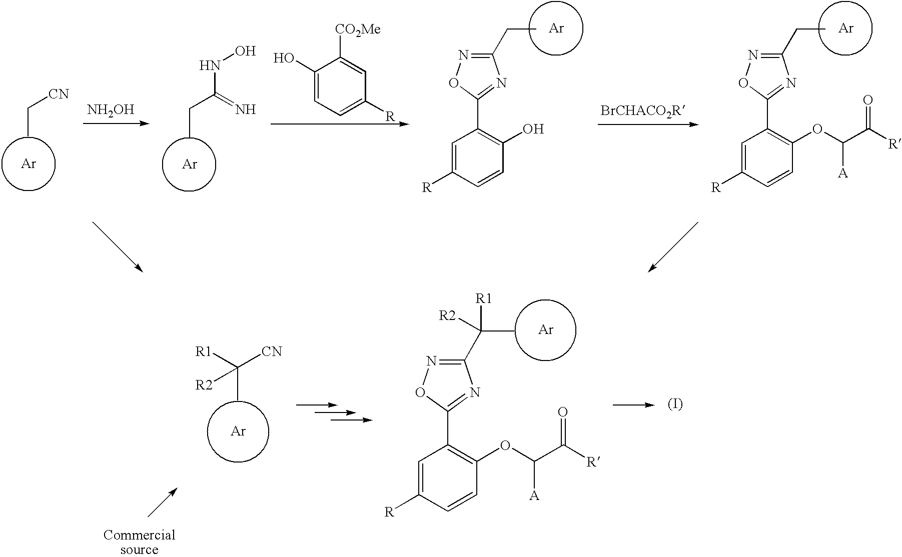

The R₂ group containing a cyclic nitrogen ring can be introduced from the corresponding ketone, obtained by standard oxidation conditions, by e.g. reductive alkylation. Optionally the ketone may be introduced in a protected form such as ketal at an earlier stage of the synthesis. Alternatively, the R₂ group may be introduced by nucleophilic displacement of a corresponding compound containing a leaving group Lg such as chlorine, bromine, mesyl, or tosyl as illustrated. Optionally the leaving group may be introduced in a protected form at an earlier stage of the synthesis, e.g. from a cyanohydrin that is protected as acetal prior to the transformation of the nitrile into the oxadiazole. Another approach utilise an aldehyde function on the oxadiazole that is functionalised by a suitable metalorganic species containing the Ar moiety followed by conversion of the resulting hydroxyl group into the nitrogen containing ring.

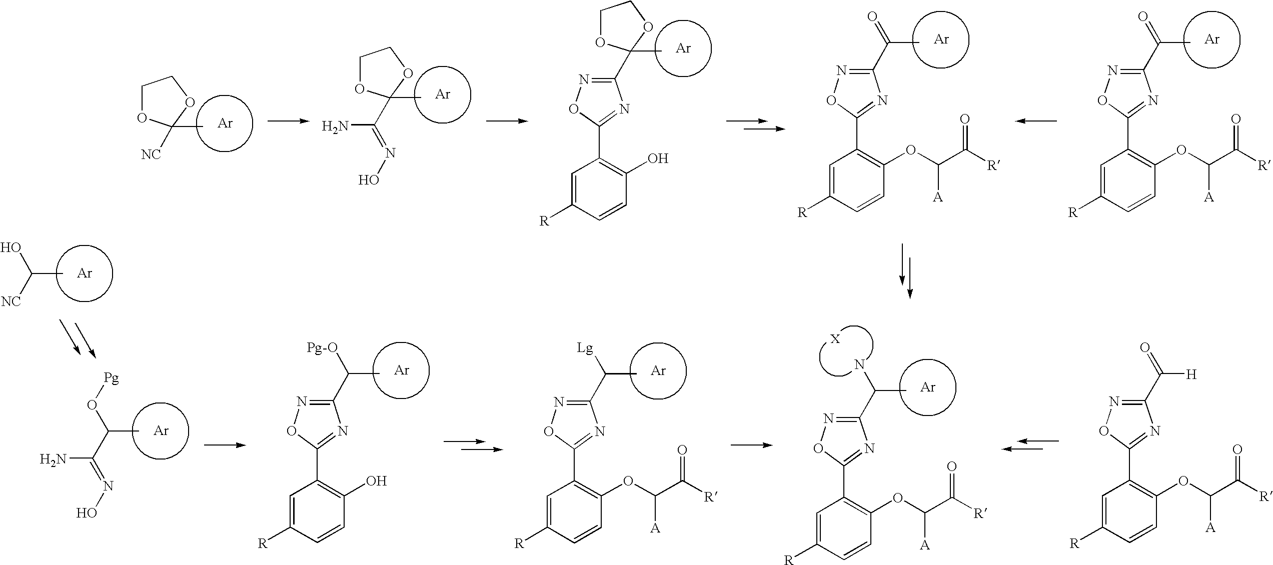

The compounds having R₁ and R₂ adjoined in a common ring can for example be introduced by the following strategy where Lg denotes a leaving group such as chlorine, bromine, mesyl or tosyl and Pg denotes a protecting group when needed, such as t-Boc for nitrogen. After deprotection the X₁ group can be further manipulated by standard reactions, e.g. for X₁ being secondary amine, alkylation or acylation with a sulfonyl chlorides or acyl chlorides to produce alkylated amines, sulfonamides or carboxamides, respectively.

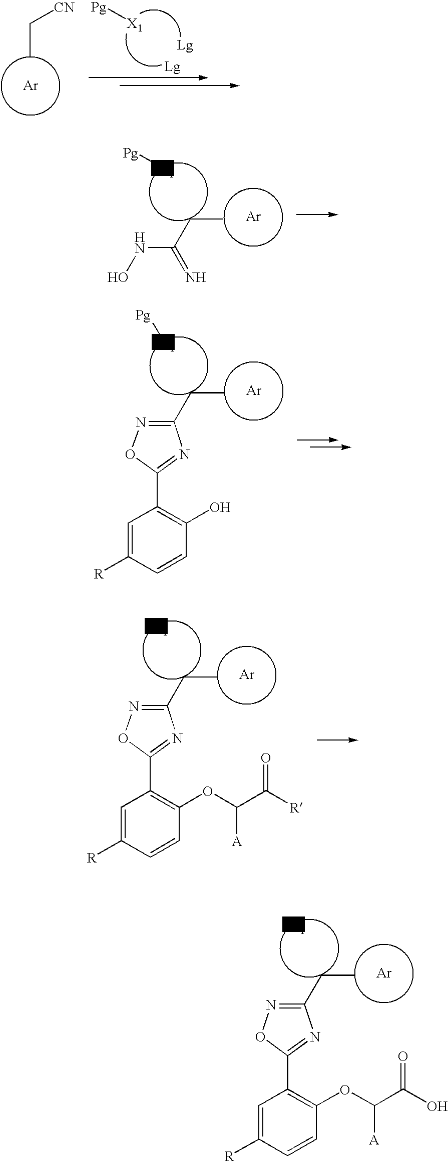

The following Examples illustrate the preparation of compounds with which this invention is concerned.

### General Comments:

Microwave chemistry was performed in a Personal Chemistry Emrys Optimizer. NMR spectra were obtained on a Bruker Avance AMX 300 MHz instrument. LC/MS was performed on an Agilent 1100-series instrument. LC/MS methods are as follows: An10p8: Column: XTerra MS C18; Flow: 1.0 mL/min; Gradient: 0-5 min: 15-100% MeCN in water, 5-7½ min: 100% MeCN; Modifier: 5 mM ammonium formate; MS-ionisation mode: API-ES (pos.). An10n8: Column: XTerra MS C18; Flow: 1.0 mL/min; Gradient: 0-5 min: 15-100% MeCN in water, 5-7½ min: 100% MeCN; Modifier: 5 mM ammonium formate; MS-ionisation mode: API-ES (neg.). TFA20p5: Column: Gemini 5μ C18 50×2.00 mm; Flow: 1.2 mL/min; Gradient: 0-3½ min: 10-95% MeCN in water, 3½/4½ min: 95% MeCN; Modifier: 0.1% TFA; MS-ionisation mode: API-ES (pos.).

### General Synthetic Route I

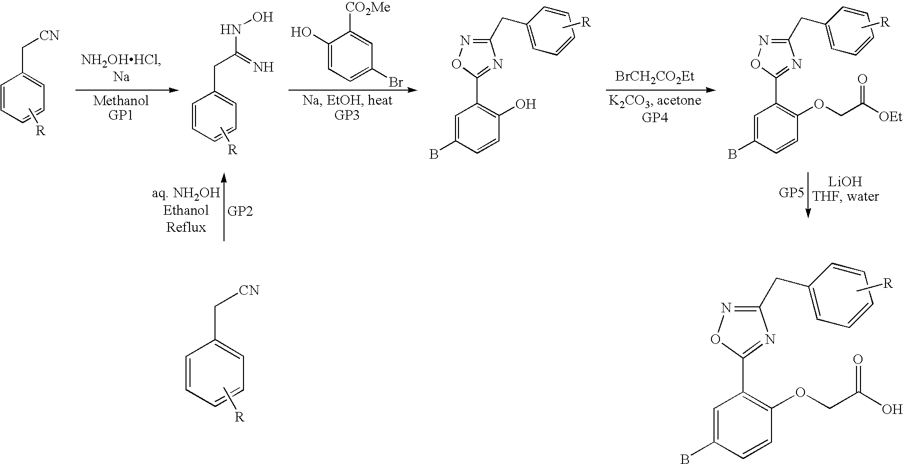

### General Synthetic Route II

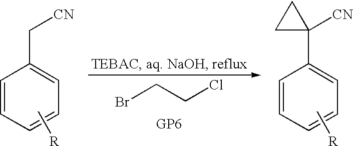

### General Synthetic Route III

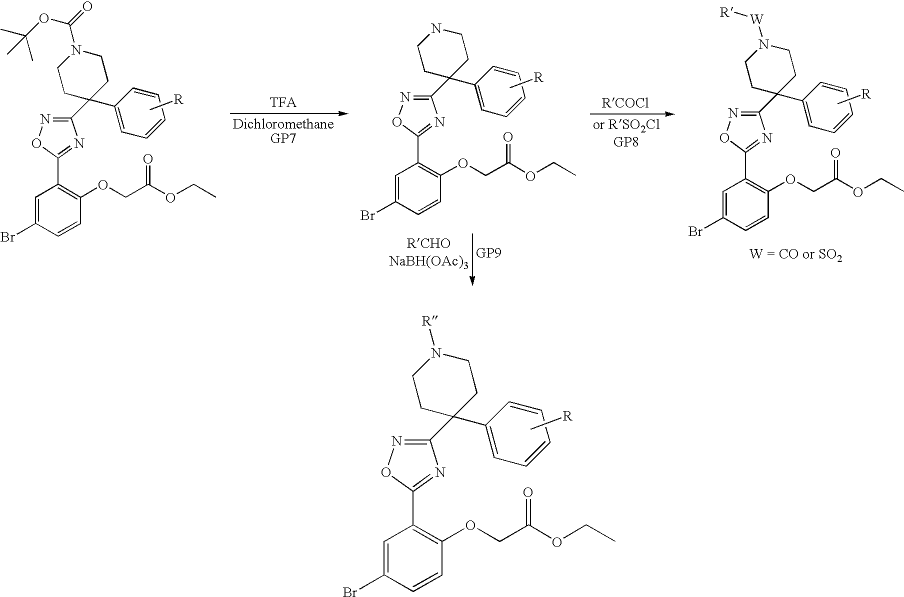

### General Procedure 1 (GPI)

### Synthesis of Amidoximes

Sodium (1.25 mmol) was added to dry methanol (1 ml) to give solution A. Hydroxylamine hydrochloride (1.2 mmol) was dissolved in dry methanol (1 mL) to give solution B. Solution A and B were mixed, cooled in an ice-bath and filtered. To the filtrate was then added the nitrile (1 mmol) and the reaction mixture was stirred over night at room temperature. The solvent was removed in vacuo to give the corresponding amidoxime. The compound was purified over silica gel chromatography (EtOAc/Heptane:1/2) or used without further purification.

### General Procedure 2 (GP2)

### Synthesis of Amidoximes

To 50 ml of 96% ethanol was added the nitrile (10 mmol) and 50% hydroxylamine (40 mmol) in water. The mixture was heated to reflux for 2 hours. After cooling, the solvent was removed in vacuo and water was added and the mixture was stirred until precipitation occurred. The precipitate was filtered and dried in vacuo.

### General Procedure 3 (GP3)

### Synthesis of Oxadiazoles

To a solution of sodium (3.3 mmol) in dry ethanol (10 mL) were successively added the amidoxime (1.15 mmol), molecular sieves (1 g) and methyl benzoate (1 mmol). After stirring for 12 h under reflux, the reaction mixture was cooled and filtered through a celite pad. The celite pad was washed with methanol and CH₂Cl₂. The solvent was removed in vacuo and the residue was stirred with water. The precipitate was filtered off and dried to give the corresponding oxadiazole. The compound was purified over silica gel chromatography (EtOAc:Heptane, 1:2) or used without further purification.

### General Procedure 4 (GP4):

### Alkylation of Phenol

The phenol (0.5 mmol) in acetone (1 mL) was added ethyl bromoacetate (85 mg, 0.5 mmol) or ethyl 2-bromopropionate (91 mg, 0.5 mmol) or ethyl-2-(trifluoromethylsulfonyl)propionate (125 mg, 0.5 mmol) and K₂CO₃ (75 mg, 0.54 mmol), and the reaction mixture was stirred at room temperature for 12 h. The reaction mixture was then concentrated in vacuo and the residue was partitioned between water and ethyl acetate. The organic phase was washed with brine, dried (MgSO₄) and concentrated. The product was used directly or purified by recrystallization from MeOH or by flash chromatography.

### General Procedure 5 (GP5):

### Hydrolysis of Ester

To the ester (0.10 mmol) in THF (0.5 mL) was added LiOH.H₂O (6.3 mg, 0.15 mmol) in water (0.5 mL). The reaction was stirred at room temperature for >2 h, 3% HCl was added until pH<1, and the mixture was extracted with CH₂Cl₂. The organic phase was dried (MgSO₄) and concentrated to give the product.

### General Procedure 6 (GP6):

### Synthesis of Cyclopropyl

To a mixture of phenylacetonitrile (20 mmol) and triethylbenzylammoniumchloride (2 mmol) in 50% aqueous sodium hydroxide (10 ml), was slowly added 1-bromo-2-chloroethane (30 mmol). The mixture was refluxed for 12 hours. After cooling, the mixture was partitioned between water and ethylacetate. The organic phase was dried (Na₂SO₄), reduced in vacuum and purified by flash chromatography (EtOAc:Heptane, 1:4).

### General Procedure 7 (GP7):

### Removal of Boc Protecting Group

The Boc. Protected compound (2.6 mmol) was stirred in 10% TFA in dichloromethane (15 ml) at room temperature for 12 hours. Sat. aqueous sodium carbonate was added and dichloromethane was removed in vacuo. The residue was partitioned between water and ethylacetate. The organic phase was dried (Na₂SO₄), reduced in vacuo and purified by flash chromatography (EtOAc, then EtOAc:MeOH, 1:1).

### General Procedure 8 (GP8):

Alkylation of Piperidine Nitrogen with RCOCl or RSO2Cl

To a cooled (0° C.) mixture of the “piperidine” (0.3 mmol) and triethylamine (0.33 mmol) in dichlorometane (5 ml) was added the acid chloride or sulfonyl chloride (0.33 mmol). The reaction mixture was stirred at room temperature for 2 hours. Solvent was removed in vacuo. The residue was partitioned between water and dichloromethane. The organic phase was dried (Na₂SO₄), reduced in vacuo and purified by flash chromatography (EtOAc:Heptane, 1:1) or used without further purification.

### General Procedure 9 (GP9):

### Reductive Alkylation of Piperidine Nitrogen

To a mixture of the “piperidine” (0.2 mmol) and sodium triacetoxyborohydride (0.9 mmol) in dichloromethane (8 ml) was added formaldehyde 37% (0.9 mmol). The reaction mixture was stirred at room temperature overnight. Sat. aqueous sodium hydrogencarbonate was added and the compound extracted with dichloromethane. The organic phase was dried (Na₂SO₄), reduced in vacuo and used without further purification.

### Preparation of Intermediates

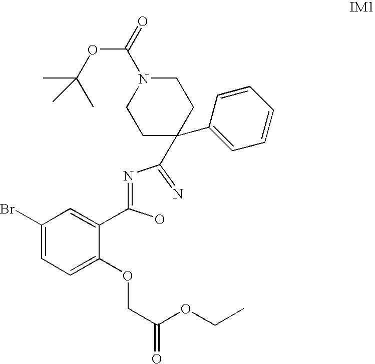

4-[5-(5-Bromo-2-ethoxycarbonylmethoxy-phenyl)-[1,2,4]oxadiazol-3-yl]-4-phenyl-piperidine-1-carboxylic acid tert-butyl ester. Title compound was prepared from 5-bromo-2-hydroxybenzoic acid methyl ester and 4-Cyano-4-phenyl-piperidine-1-carboxylic acid tert-butyl ester according to GP2, GP3 and GP4: LC/MS (tfa20p5.m) Rt 3.22 min, m/z 566 [M+H]⁻;

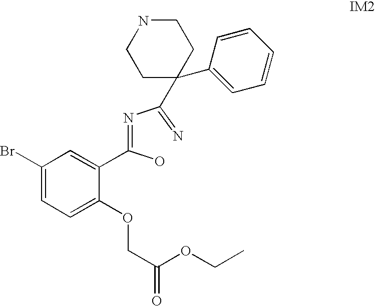

{4-Bromo-2-[3-(4-phenyl-piperidin-4-yl)-[1,2,4]oxadiazol-5-yl]-phenoxy}-acetic acid ethyl ester. Title compound was prepared from intermediate IM1 according to GP7: LC/MS (tfa20p5.m) Rt 2.194 min, m/z 502 [M+H]⁻;

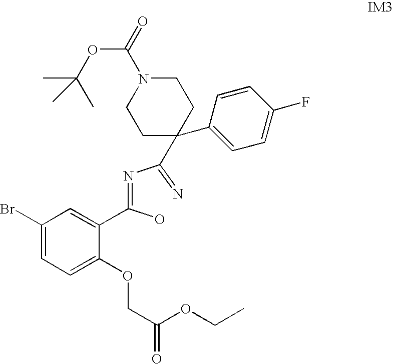

4-[5-(5-Bromo-2-ethoxycarbonylmethoxy-phenyl)-[1,2,4]oxadiazol-3-yl]-4-(4-fluoro-phenyl)-piperidine-1-carboxylic acid tert-butyl ester. Title compound was prepared from 5-bromo-2-hydroxybenzoic acid methyl ester and 4-Cyano-4-(4-fluoro-phenyl)-piperidine-1-carboxylic acid tert-butyl ester according to GP2, GP3 and GP4: LC/MS (tfa20p5.m) Rt 3.75 min, m/z 628[M+Na].

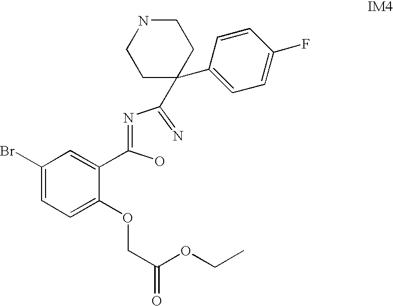

(4-Bromo-2-{3-[4-(4-fluoro-phenyl)-piperidin-4-yl]-[1,2,4]oxadiazol-5-yl}-phenoxy)-acetic acid ethyl ester. Title compound was prepared from intermediate IM3 according to GP7: LC/MS (tfa20p5.m) Rt 2.3 min, m/z 506 [M+H]⁻;

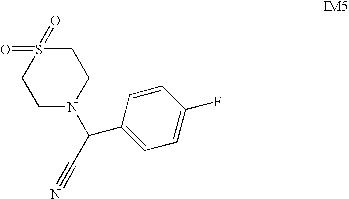

(1,1-Dioxo-1lambda*6*-thiomorpholin-4-yl)-(4-fluoro-phenyl)-acetonitrile. Title compound was prepared from 4-Fluoro-benzaldehyde and Thiomorpholine 1,1-dioxide as follow:

To a solution of Thiomorpholine 1,1-dioxide (2.52 g, 18.64 mmol) in 1N aqueous HCl (18.64 ml, 18.64 mmol) was added sodium cyanide (0.822 g, 16.78 mmol). After dissolution of sodium cyanide, a solution of 4-Fluoro-benzaldehyde (1 ml, 9.32 mmol) in acetonitrile (38 ml) was added dropwise. The reaction mixture was stirred at room temperature for 3 days. Solvent was removed in vacuo and water was added. The mixture was stirred for ˜10 minutes and the white precipitate was filtered off, washed with water and dried in vacuo to give title compound (1.74 g, 6.48 mmol, 70%). LC/MS (tfa20p5.m) Rt 2.01 min, m/z 269 [M+H]⁻; ¹H NMR (CDCl₃): δ 3.12 (m, 8H), 4.94 (s, 1H), 7.16 (t, 2H), 7.53 (dd, 2H).

### EXAMPLES

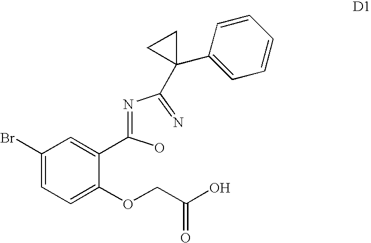

{4-Bromo-2-[3-(1-phenylcyclopropyl)-[1,2,4]oxadiazol-5-yl]phenoxy}acetic acid. Title compound was prepared from 5-bromo-2-hydroxy-benzoic acid methyl ester and 1-phenyl-1-cyclopropanecarbonitrile according to GP1, GP3, GP4 and GP5: LC/MS (an10n8) Rt.2.756 m/z 413.4 [M−H]⁻; ¹H NMR (DMSO-d₆): δ 0.92 (m, 2H), 1.11 (m, 2H), 4.38 (s, 2H), 6.63-6.66 (d, 1H), 6.79-6.88 (m, 3H), 6.93-6.95 (m, 2H), 7.25-7.29 (dd, 1H), 7.50-7.51 (d, 1H).

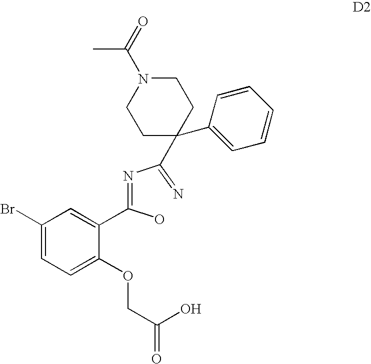

{2-[3-(1-Acetyl-4-phenyl-piperidin-4-yl)-[1,2,4]oxadiazol-5-yl]-4-bromo-phenoxy}-acetic acid. Title compound was prepared from example D5 and acetic anhydride as follow:

A suspension of example D5 (0.04 mmol) in acetic anhydride (0.5 ml) was heated to 50° C. for 1 hour. Solvent was removed in vacuo to give a colourless gum. Water was added and the mixture was stirred vigorously until of a fine precipitate was formed. The precipitate was filtered off, washed with water and dried in vacuo to give title compound: LC/MS (an10p8.n) Rt 2,507 min, m/z 500 [M−H]⁻; ¹H NMR (DMSO): δ 2.0 (s, 3H), 1.95-2.3 (m, 2H), 2.6-2.7 (m, 2H), 2.8-2.9 (m, 1H), 3.2-3.3 (m, 1H), 3.75-3.85 (m, 1H), 4.15-4.3 (m, 1H), 4.9 (s, 3H), 7.15-7.19 (d, 1H), 7.21-7.28 (1H), 7.31-7.43 (m, 4H), 7.75-7.81 (dd, 1H), 8.05-8.07 (d, 1H).

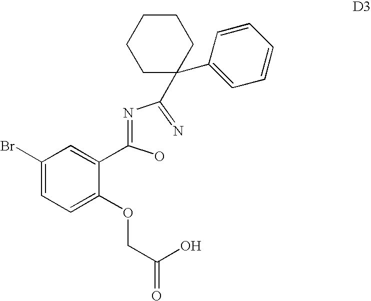

{4-Bromo-2-[3-(1-phenyl-cyclohexyl)-[1,2,4]oxadiazol-5-yl]-phenoxy}-acetic acid. Title compound was prepared from 5-bromo-2-hydroxybenzoic acid methyl ester and 1-Phenyl-cyclohexanecarbonitrile according to GP1, GP3, GP4 and GP5: LC/MS (an10p8.m) Rt 3.21 min, m/z 457 [M+H]⁻; ¹H NMR (DMSO): δ 1.3-1.7 (m, 6H), 2.0-2.1 (m, 2H), 2.5-2.6 (m, 2H), 4.9 (m, 2H), 7.13-7.25 (m, 2H), 7.28-7.41 (m, 4H), 7.74-7.80 (dd, 1H), 8.00-8.03 (d, 1H)

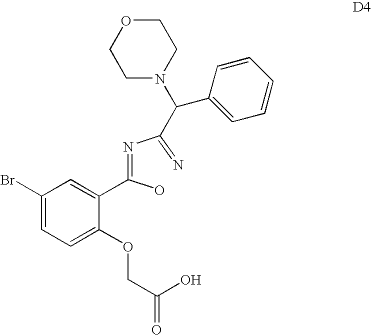

{4-Bromo-2-[3-morpholin-4-yl-phenyl-methyl)-[1,2,4]oxadiazol-5-yl]-phenoxy}-acetic acid. Title compound was prepared from 5-bromo-2-hydroxybenzoic acid methyl ester and Morpholin-4-yl-phenyl-acetonitrile according to GP1, GP3, GP4 and GP5: LC/MS (an10n8.m) Rt 2.39 min, m/z 474 [M−H]⁻; ¹H NMR (DMSO): δ 1.9 (s, 1H), 2.3-2.5 (m, 3H), 3.6 (s, 4H), 4.85 (s, 1H), 4.9 (s, 2H), 7.13-7.2 (d, 1H), 7.27-7.45 (m, 4H), 7.5-7.6 (m, 2H), 7.75-7.83 (d, 1H), 8.05-8.1 (s, 1H).

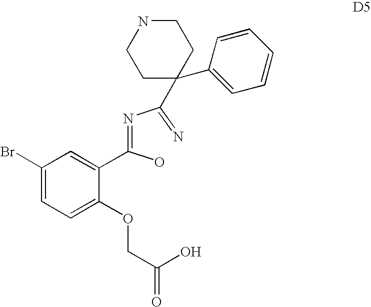

{4-Bromo-2-[3-(4-phenyl-piperidin-4-yl)-[1,2,4]oxadiazol-5-yl]-phenoxy}-acetic acid. Title compound was prepared from intermediate IM2 and lithium hydroxide as follow:

To the ester (0.10 mmol) in THF (0.5 mL) was added LiOH.H₂O (6.3 mg, 0.15 mmol) in water (0.5 mL). The reaction was stirred at room temperature for >2 h, aq. HCl was added until precipitation occurred. The precipitate was filtered off and dried in vacuo to give title compound.

LC/MS (an10n8.m) Rt 2.33 min, m/z 458 [M−H]⁻; ¹H NMR (DMSO): δ 2.3 (m, 2H), 2.8 (m, 2H), 2.95 (m, 2H), 3.2 (m, 2H), 4.4 (s, 2H), 6.9 (s, 1H), 7.3 (m, 1H), 7.4 (m, 4H), 7.65 (d, 1H), 8.0 (s, 1H),

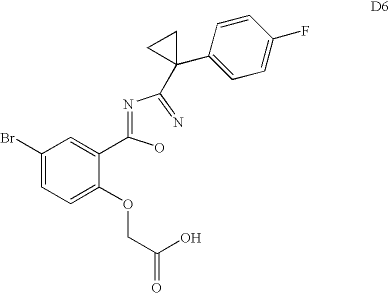

(4-Bromo-2-{3-[1-(4-fluoro-phenyl)-cyclopropyl]-[1,2,4]oxadiazol-5-yl}-phenoxy)-acetic acid. Title compound was prepared from 5-bromo-2-hydroxybenzoic acid methyl ester and 1-(4-Fluoro-phenyl)-cyclopropanecarbonitrile according to GP6, GP2, GP3, GP4 and GP5 LC/MS (tfa20p5.m) Rt 3.09 min, m/z 436 [M+H]⁻; ¹H NMR (DMSO): δ 1.45 (m, 2H), 1.65 (m, 2H), 4.9 (s, 2H), 7.1-7.2 (m, 3H), 7.4-7.55 (m, 2H), 7.8 (dd, 1H), 8.0 (d, 1H).

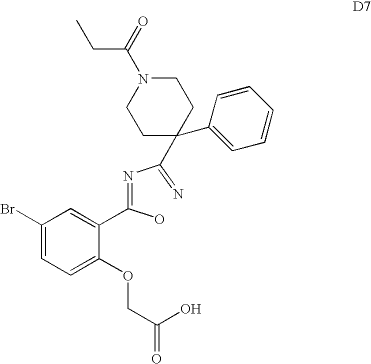

{4-Bromo-2-[3-(4-phenyl-1-propionyl-piperidin-4-yl)-[1,2,4]oxadiazol-5-yl]-phenoxy}-acetic acid. Title compound was prepared from intermediate IM2 and Propionyl chloride according to GP8 and GP5 LC/MS (tfa20p5.m) Rt 2.78 min, m/z 514 [M+H]⁻; ¹H NMR (DMSO): δ 0.95-1.0 (t, 3H), 2.0-2.2 (m, 2H), 2.3-2.4 (m, 2H), 2.55-2.70 (m, 2H), 2.8-2.95 (m, 1H), 3.15-3.3 (m, 1H), 3.75-3.9 (m, 1H), 4.15-4.3 (m, 1H), 4.85 (s, 2H), 7.13-7.18 (d, 1H), 7.2-7.28 (m, 1H), 7.31-7.43 (m, 4H), 7.74-7.80 (dd, 1H), 8.03-8.06 (d, 1H).

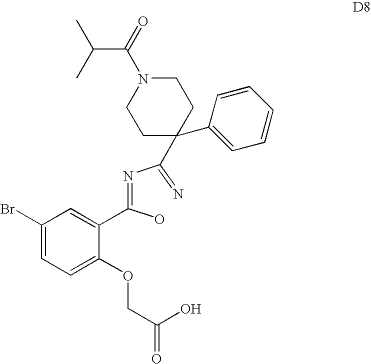

{4-Bromo-2-[3-(1-isobutyryl-4-phenyl-piperidin-4-yl)-[1,2,4]oxadiazol-5-yl]-phenoxy}-acetic acid. Title compound was prepared from intermediate IM2 and Isobutyryl chloride according to GP8 and GP5: LC/MS (tfa20p5.m) Rt 2.947 min, m/z 528 [M+H]⁻; ¹H NMR (DMSO): δ 0.95 1.05 (m, 6H), 1.95-2.02 (m, 2H), 2.6-2.75 (m, 2H), 2.8-2.95 (m, 2H), 3.2-3.3 (m, 1H), 3.85-3.95 (m, 1H), 4.2-4.3 (m, 1H), 4.85 (s, 2H), 7.1-7.17 (d, 1H), 7.2-7.28 (m, 1H), 7.31-7.44 (m, 4H), 7.73-7.78 (dd, 1H), 8.5 (d, 1H).

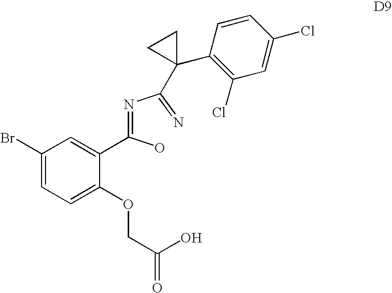

(4-Bromo-2-{3-[1-(2,4-dichloro-phenyl)-cyclopropyl]-[1,2,4]oxadiazol-5-yl}-phenoxy)-acetic acid. Title compound was prepared from 5-bromo-2-hydroxybenzoic acid methyl ester and 1-(2,4-Dichloro-phenyl)-cyclopropanecarbonitrile according to GP6, GP2, GP3, GP4 and GP5: LC/MS (tfa20p5.m) Rt 3.483 min, m/z 485 [M+H]⁻; ¹H NMR (DMSO): δ 1.4-1.5 (m, 2H), 1.7-1.8 (m, 2H), 4.6 (s, 2H), 7.0-7.04 (d, 1H), 7.44-7.49 (dd, 1H), 7.58-7.62 (d, 1H), 7.66-7.68 (d, 1H), 7.69-7.75 (dd, 1H), 7.96-7.99 (d, 1H).

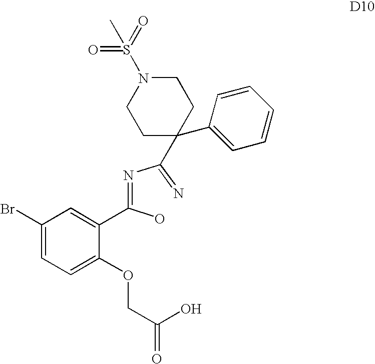

{4-Bromo-2-[3-(1-methanesulfonyl-4-phenyl-piperidin-4-yl)-[1,2,4]oxadiazol-5-yl]-phenoxy}-acetic acid. Title compound was prepared from intermediate IM2 and Methanesulfonyl chloride according to GP8 and GP5:LC/MS (tfa20p5.m) Rt 2.725 min, m/z 538 [M+H]⁻; ¹H NMR (DMSO): δ 2.2-2.3 (m, 2H), 2.7-3.0 (m, 7H), 3.5-3.6 (m, 2H), 4.9 (s, 2H), 7.14-7.19 (d, 1H), 7.21-7.29 (m, 1H), 7.32-7.44 (m, 4H), 7.75-7.80 (dd, 1H), 8.05-8.08 (d, 1H).

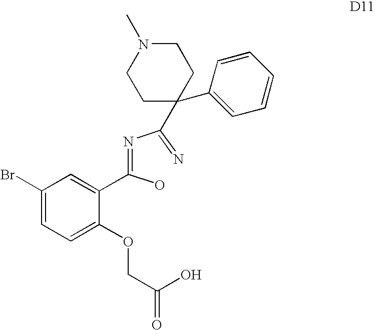

{4-Bromo-2-[3-(1-methyl-4-phenyl-piperidin-4-yl)-[1,2,4]oxadiazol-5-yl]-phenoxy}-acetic acid. Title compound was prepared from intermediate IM2 and formaldehyde according to GP9 and GP5: LC/MS (tfa20p5.m) Rt 1.849 min, m/z 474[M+H]⁻; ¹H NMR (DMSO): δ 2.25-2.4 (m, 5H), 2.5 (m, 2H), 2.65-2.75 (m, 2H), 2.95-3.05 (m, 2H), 4.7 (s, 2H), 7.03-7.1 (d, 1H), 7.2-7.29 (m, 1H), 7.3-7.45 (m, 4H), 7.67-7.73 (dd, 1H), 8.01-8.04 (d, 1H).

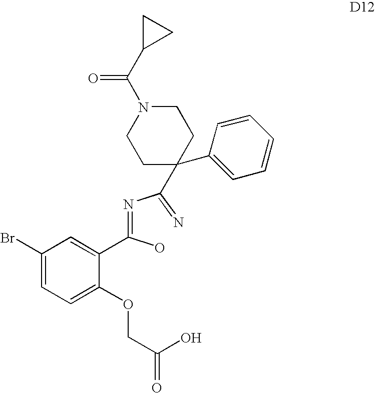

{4-Bromo-2-[3-(1-cyclopropanecarbonyl-4-phenyl-piperidin-4-yl)-[1,2,4]oxadiazol-5-yl]-phenoxy}-acetic acid. Title compound was prepared from intermediate IM2 and Cyclopropanecarbonyl chloride according to GP8 and GP5: LC/MS (tfa20p5.m) Rt 2.82 min, m/z 528 [M+H]⁻; ¹H NMR (DMSO): δ 0.68-0.70 (m, 4H), 2.00-2.27 (m, 2H), 2.72 (m, 2H), 3.1 (m, 1H), 4.08 (m, 1H), 4.18 (m, 2H), 4.91 (s, 2H), 7.15-7.18 (d, 1H), 7.25-7.27 (m, 1H), 7.32-7.42 (m, 4H), X (7.76-7.80 (dd, 1H), 8.05-8.06 (d, 1H),

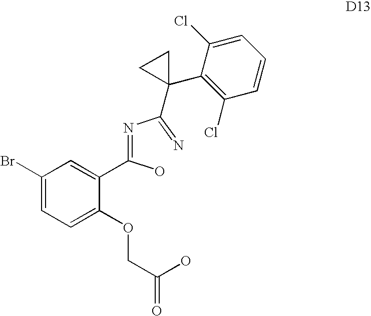

(4-Bromo-2-{3-[1-(2,6-dichloro-phenyl)-cyclopropyl]-[1,2,4]oxadiazol-5-yl}-phenoxy)-acetic acid. Title compound was prepared from 5-bromo-2-hydroxybenzoic acid methyl ester and 1-(2,6-Dichloro-phenyl)-cyclopropanecarbonitrile according to GP6, GP2, GP3, GP4 and GP5: LC/MS (tfa20p5.m) Rt 3.313 min, m/z 485 [M+H]⁻; ¹H NMR (DMSO): δ 1.54-1.58 (m, 2H), 1.93-1.98 (m, 2H), 4.89 (s, 2H), 7.15-7.19 (d, 1H), 7.39-7.55 (m, 2H), 7.77-7.81 (dd, 1H), 8.04-8.05 (d, 1H).

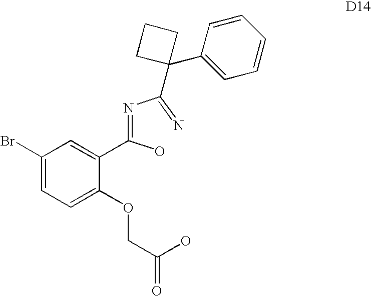

{4-Bromo-2-[3-(1-phenyl-cyclobutyl)-[1,2,4]oxadiazol-5-yl]-phenoxy}-acetic acid. Title compound was prepared from 5-bromo-2-hydroxybenzoic acid methyl ester and 1-Phenyl-cyclobutanecarbonitrile according to GP6, GP2, GP3, GP4 and GP5: LC/MS (an10p.8.m) Rt 2.90 min, m/z 429 [M+H]⁻; ¹H NMR (DMSO): δ 1.97 (m, 1H), 2.08 (m, 1H), 2.68-2.72 (m, 2H), 2.87 (m, 2H), 4.90 (s, 2H), 7.14-7.17 (d, 1H), 7.22-7.26 (m, 1H), 7.35-7.36 (m, 4H), 7.75-7.79 (dd, 1H), 8.00-8.02 (d, 1H), 13.17 (s, 1H).

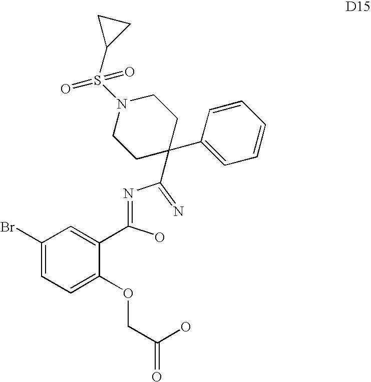

{4-Bromo-2-[3-(1-cyclopropanesulfonyl-4-phenyl-piperidin-4-yl)-[1,2,4]oxadiazol-5-yl]-phenoxy}-acetic acid. Title compound was prepared from intermediate IM2 and Cyclopropanesulfonyl chloride according to GP8 and GP5:LC/MS (tfa20p5.m) Rt 2.9 min, m/z 564 [M+H]⁻; ¹H NMR (DMSO): δ 0.98 (m, 4H) 2.2 (m, 2H), 2.56 (m, 1H), 2.75 (m, 2H), 3.04 (m, 2H), 3.58 (m, 2H), 4.9 (s, 2H), 7.15-7.18 (d, 1H), 7.25-7.28 (m, 1H), 7.33-7.42 (m, 4H), 7.77-7.78 (dd, 1H), 8.06-8.07 (d, 1H), 13.2 (s, 1H).

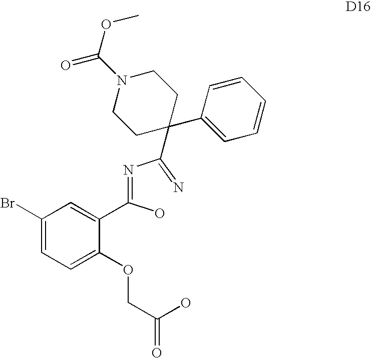

4-[5-(5-Bromo-2-carboxymethoxy-phenyl)-[1,2,4]oxadiazol-3-yl]-4-phenyl-piperidine-1-carboxylic acid methyl ester. Title compound was prepared from intermediate IM2 and Methyl chloroformate according to GP8 and GP5:LC/MS (tfa20p5.m) Rt 2.8 min, m/z 516 [M+H]⁻; ¹H NMR (DMSO): δ 2.1 (m, 2H), 2.65 (m, 2H), 3.08 (m, 2H), 3.6 (s, 3H), 3.9 (m, 2H), 4.9 (s, 2H), 7.15-7.19 (d, 1H), 7.21-7.28 (m, 1H), 7.3-7.42 (m, 4H), 7.76-7.81 (dd, 1H), 8.30-8.50 (d, 1H).

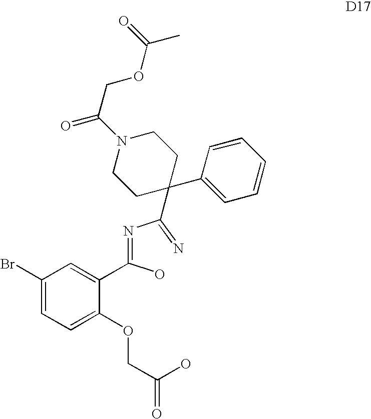

(2-{3-[1-(2-Acetoxy-acetyl)-4-phenyl-piperidin-4-yl]-[1,2,4]oxadiazol-5-yl}-4-bromo-phenoxy)-acetic acid. Title compound was prepared from D5 and Acetoxyacetyl chloride as follows:

A mixture of D5 (230 mg, 0.5 mmol), acetoxyacetyl chloride (120 ul, 1.1 mmol) and triethylamine (160 ul, 1.1 mmol) in tetrahydrofuran (10 ml) was stirred at 0° C. for 2 hours, under an argon atmosphere. The reaction mixture was concentrated in vacuo. The residue was partitioned between dichloromethane and water. The phases were separated and the organic phase was dried over MgSO₄ and concentrated in vacuo. The residue was purified over silica gel chromatography (eluent: CH₂Cl₂/MeOH: 10/1) to give title compound (41 mg, 0.07 mmol, 15%). LC/MS (tfa20p5.m) Rt 2.5 min, m/z 560[M+H]⁻; ¹H NMR (DMSO): δ 2.0 (m, 4H), 2.25 (m, 1H), 2.65 (m 2H), 2.9 (m, 1H), 3.3 (m, 1H), 3.7 (m, 1H), 4.15 (m, 1H), 4.42 (s, 2H), 4.8 (d, 2H), 6.95-6.99 (d, 1H), 7.12-7.28 (m, 1H), 7.31-7.41 (m, 4H), 7.65-7.71 (dd, 1H), 7.98-8.00 (d, 1H).

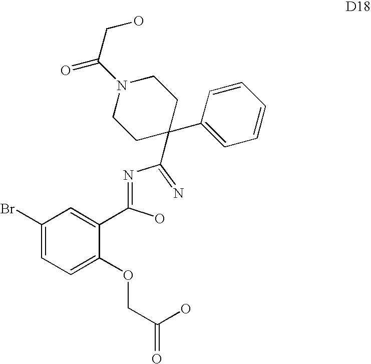

(4-Bromo-2-{3-[1-(2-hydroxy-acetyl)-4-phenyl-piperidin-4-yl]-[1,2,4]oxadiazol-5-yl}-phenoxy)-acetic acid. Title compound was prepared from intermediate IM2 and Acetoxyacetyl chloride according to GP8 and GP5:LC/MS (tfa20p5.m) Rt 2.4 min, m/z 516 [M+H]⁻;

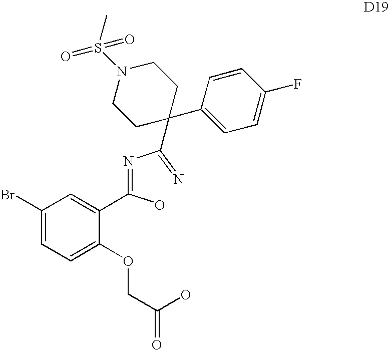

(4-Bromo-2-{3-[4-(4-fluoro-phenyl)-1-methanesulfonyl-piperidin-4-yl]-[1,2,4]oxadiazol-5-yl}-phenoxy)-acetic acid. Title compound was prepared from intermediate IM4 and Methanesulfonyl chloride according to GP8 and GP5: LC/MS (tfa20p5.m) Rt 2.8 min, m/z 556 [M+H]⁻; ¹H NMR (DMSO): δ 2.35 (m, 2H), 2.7-3.0 (m 7H), 3.55 (m, 2H), 4.9 (s, 2H), 7.14-7.22 (m, 3H), 7.42-7.50 (m, 2H), 7.77-7.82 (dd, 1H), 8.08-8.09 (d, 1H).

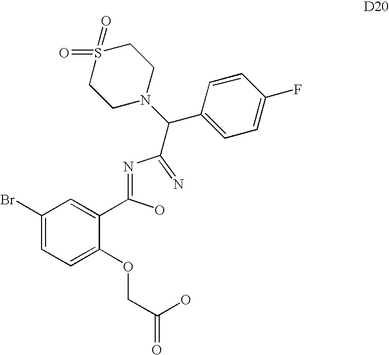

(4-Bromo-2-{3-[(1,1-dioxo-1lambda*6*-thiomorpholin-4-yl)-(4-fluoro-phenyl)-methyl]-[1,2,4]oxadiazol-5-yl}-phenoxy)-acetic acid. Title compound was prepared from 5-bromo-2-hydroxybenzoic acid methyl ester and intermediate IM5 according to GP1, GP3, GP4 and GP5: LC/MS (tfa20p5.m) Rt 2.62 min, m/z 540 [M+H]⁻; ¹H NMR (CDCl₃): δ 3.12 (m, 8H), 4.78 (s, 2H), 5.14 (s, 1H), 6.97 (d, 1H), 7.10 (t, 2H), 7.48 (dd, 2H), 7.74 (dd, 1H), 8.23 (s, 1H).

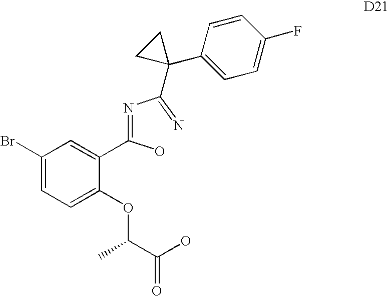

(S)-2-(4-Bromo-2-{3-[1-(4-fluoro-phenyl)-cyclopropyl]-[1,2,4]oxadiazol-5-yl}-phenoxy)-propionic acid. Title compound was prepared from 5-bromo-2-hydroxybenzoic acid methyl ester, 1-(4-Fluoro-phenyl)-cyclopropanecarbonitrile and ethyl(R)-2-(trifluoromethylsulfonyl)propionate according to GP6, GP2, GP3, GP4 and GP5: LC/MS (tfa20p5.m) Rt 3.20 min, m/z 447[M+H]⁻; ¹H NMR (CDCl₃): δ, 1.36-1.38 (m, 2H), 1.61-1.65 (m, 2H), 1.69-1.72 (d, 3H), 3.65-3.67 (m, 1H), 6.88-6.91 (d, 1H), 6.96-7.01 (m, 2H), 7.37-7.41 (m, 2H), 7.60-7.63 (dd, 1H), 8.06-8.07 (d, 1H).

### Biological Assays

### Materials and Methods

Generation/origin of the cDNA Constructs. The coding sequence of the human CRTH2 receptor (genbank accession no NM_{—}004778) was amplified by PCR from a human hippocampus cDNA library and inserted into the pcDNA3.1(+) expression vector (invitrogen) via 5′ HindIII and 3′ EcoRI. To generate a CRTH2-Renilla luciferase (CRTH2-Rluc) fusion protein, the CRTH2 coding sequence without a STOP codon and Rluc were amplified, fused in frame by PCR and subcloned into the pcDNA3.1(+)Zeo expression vector (invitrogen). Human β-arrestin2 (β-arr2) N-terminally tagged with GFP² (βarr2-GFP²) and Renilla luciferase were purchased from BioSignal Packard Inc, (Montreal, Canada). The sequence identity of the construct was verified by restriction endonuclease digests and sequencing in both directions on an ABI Prism (Applied Biosystems, Foster City, Calif.).

Cell Culture and Transfection. COS-7 cells were grown in Dulbecco's modified Eagle's medium (DMEM) 1885 supplemented with 10% fetal bovine serum, 100 units/ml penicillin, 1000 μg/ml streptomycin, and kept at 37° C. in a 10% CO₂ atmosphere. HEK293 cells were maintained in Minimum Essential medium (MEM) supplemented with 10% (v/v) heat inactivated fetal calf serum (HIFCS), 2 mM Glutamax™-I, 1% non essential amino acids (NEAA), 1% sodium pyruvate and 10 μg/ml gentamicin. For binding experiments, COS7 cells were transiently transfected with the CRTH2 receptor using a calcium phosphate-DNAcoprecipitation method with the addition of chloroquine (as described by Holst et al., 2001↓). To perform the functional Bioluminescence Resonance Energy Transfer (BRET) assays, a HEK293 cell clone stably expressing Parr2-GFP² and CRTH2-Rluc was generated (CRTH2-HEK293 cells).

Binding assay. 24 h after transfection COS-7 cells were seeded into 96 well plates at a density of 30.000 cells/well. Competition binding experiments on whole cells were then performed about 18-24 h later using 0.1 nM [³H]PGD2 (NEN, 172 Ci/mmol) in a binding buffer consisting of HBSS (GIBCO) and 10 mM HEPES. Competing ligands were diluted in DMSO which was kept constant at 1% (v/v) of the final incubation volume. Total and nonspecific binding were determined in the absence and presence of 10 μM PGD2. Binding reactions were routinely conducted for 3 h at 4° C. and terminated by 2 washes (100 μl each) with ice cold binding buffer. Radioactivity was determined by liquid scintillation counting in a TOPCOUNTER (Packard) following over night incubation in Microscint 20. Stable HEK293 cells were seeded at a density of 30.000 cells/well 18-24 h prior to the binding assay which was performed essentially as described for COS7 cells above. Determinations were made in duplicates.

BRET assay. Functional BRET assays were performed on HEK293 cells stably expressing human CRTH2-Rluc and GFP²-β-arr2. Prior to their use in the BRET assay cells were detached and re-suspended in D-PBS with 1000 mg/L L-Glucose at a density of 2×10⁶ cells/mL. DeepBlueC™ was diluted to 50 μM in D-PBS with 1000 mg/L L-Glucose (light sensitive). 100 μL of cell suspension was transferred to wells in a 96-well microplate (white OptiPlate) and placed in the Mithras LB 940 instrument (BERTHOLD TECHNOLOGIES, Bad Wildbad, Germany). 12 μL/well agonist was then injected by injector 1 and 10 μL/well DeepBlueC™ was injected simultaneously by injector 2. Five seconds after the injections the light output from the well was measured sequentially at 400 nm and 515 nm, and the BRET signal (mBRET ratio) was calculated by the ratio of the fluorescence emitted by GFP²-β-arr2 (515 nm) over the light emitted by the receptor-Rluc (400 nm). Antagonists were added before placing the microplates into the Mithras LB 940 and allowed to incubate for 15 minutes prior to the addition of agonist and DeepBlueC™. Compounds were dissolved in DMSO and the final DMSO concentration was kept constant at 1% in the assay.

Human eosinophil shape change assay. Blood was sampled from healthy volunteers according to a protocol approved by the Ethics Committee of the University of Graz and processed as described previously (Bohm et al., 2004). Preparations of polymorphonuclear leukocytes (containing eosinophils and neutrophils) were prepared by dextran sedimentation of citrated whole blood and Histopaque gradients. The resulting cells were washed and resuspended in assay buffer (comprising PBS with Ca²⁺/Mg²⁺ supplemented with 0.1% BSA, 10 mM HEPES and 10 mM glucose, pH 7.4) at 5×10⁶ cells/mL. Cells were incubated with the antagonists or vehicle (PBS or DMSO) for 10 min at 37° C. and then stimulated with various concentration of the agonists (PGD2 or eotaxin) for 4 min at 37° C. To stop the reaction, samples were transferred to ice and fixed with 250 μL of fixative solution. Samples were immediately analyzed on a FACSCalibur flow cytometer (Becton Dickinson) and eosinophils were identified according to their autofluorescence in the FL-1 and FL-2 channels. Shape change responses were quantified as percentage of the maximal response to PGD2 or eotaxin in the absence of an antagonist.

### Materials

Tissue culture media and reagents were purchased from the Gibco invitrogen corporation (Breda, Netherlands). PGD2 was obtained from Cayman and [3H]PGD2 from NEN.

### Data Analysis

Curve analysis was performed with the GraphPadPrism software 3.0 (Graphpad Prism Inc., San Diego, USA) and IC₅₀ values were calculated as a measure of the antagonistic potencies.

### REFERENCES

Hoist B, Hastrup H, Raffetseder U, Martini L, Schwartz T W. Two active molecular phenotypes of the tachykinin NK1 receptor revealed by G-protein fusions and mutagenesis. J Biol. Chem. 2001 Jun. 8; 276(23):19793-9. Epub 2001 Feb. 22.

### Biological Data:

Compounds were tested in the receptor binding assay and the functional antagonist assay described below, and their IC₅₀ values were assessed. The compounds are grouped in three classes:

A: IC₅₀ value lower than 0.5 μM
B: IC₅₀ value between 0.5 μM and 5 μM
C: IC₅₀ value higher than 5 μM

Table 1 gives the biological test results for the compounds synthesised above

TABLE 1

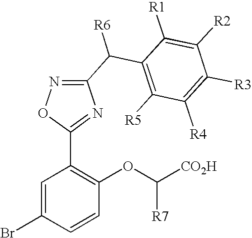

| | | | | | | | | Bind- | An- |
|---|---|---|---|---|---|---|---|---|---|
| | | | | | | | | ing | tag. |
| | R1 | R2 | R3 | R4 | R5 | R6 | R7 | IC₅₀ | IC₅₀ |
| D1 | H | H | H | H | H | 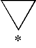 | H | A | A |
| D2 | H | H | H | H | H | 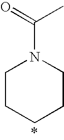 | H | A | A |
| D3 | H | H | H | H | H | 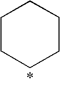 | H | A | A |
| D4 | H | H | H | H | H | 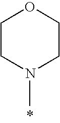 | H | A | A |
| D5 | H | H | H | H | H | 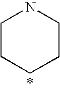 | H | A | B |
| D6 | H | H | F | H | H | 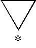 | H | A | A |
| D7 | H | H | H | H | H | 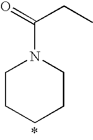 | H | A | A |
| D8 | H | H | H | H | H | 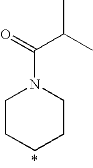 | H | A | A |
| D9 | Cl | H | Cl | H | H | 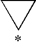 | H | A | A |
| D10 | H | H | H | H | H | 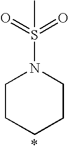 | H | A | A |
| D11 | H | H | H | H | H | 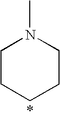 | H | A | A |
| D12 | H | H | H | H | H | 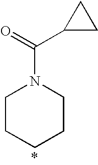 | H | A | A |
| D13 | Cl | H | H | H | Cl | 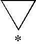 | H | A | A |
| D14 | H | H | H | H | H | 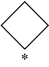 | H | A | A |
| D15 | H | H | H | H | H | 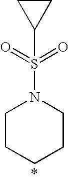 | H | A | A |
| D16 | H | H | H | H | H | 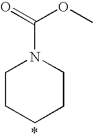 | H | A | A |
| D17 | H | H | H | H | H | 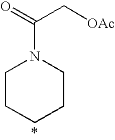 | H | A | A |
| D18 | H | H | H | H | H | 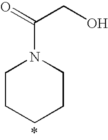 | H | A | A |
| D19 | H | H | F | H | H | 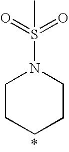 | H | A | A |
| D20 | H | H | F | H | H | 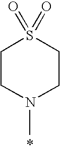 | H | A | A |
| D21 | H | H | F | H | H | 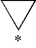 | (S) CH₃ | A | A |

1. A compound of formula (I) or a salt, hydrate or solvate thereof other than {4-bromo-2-[3-(1-phenylcyclopropyl)-[1,2,4]oxadiazol-5-yl]phenoxy}-acetic acid or a salt, hydrate or solvate thereof: 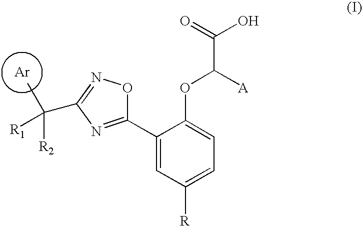
wherein
R₁ is hydrogen or methyl and R₂ is optionally substituted cycloalkyl, or optionally substituted non-aromatic heterocyclyl having 4 to 6 ring atoms; or R₁ and R₂, taken together with the carbon atom to which they are attached form an optionally substituted cycloalkyl, or optionally substituted non-aromatic heterocyclyl ring having 4 to 6 ring atoms;
R is hydrogen or an optional substituent;
the phenyl ring containing the substituent R is optionally substituted by 1, 2 or 3 optional substituents;
A is hydrogen or C₁-C₃ alkyl;
ring Ar is an optionally substituted phenyl or 5- or 6-membered monocyclic heteroaryl ring.

2. A compound as claimed in claim 1 wherein R₁ is hydrogen or methyl, and R₂ is optionally substituted cyclopropyl, cyclobutyl, cyclopentyl, or cyclohexyl.

3. A compound as claimed in claim 1 wherein R₁ is hydrogen or methyl, and R₂ is cyclopropyl.

4. A compound as claimed in claim 1 wherein R₁ is hydrogen or methyl, and R₂ is a radical of formula 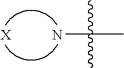
wherein the ring contains 4 to 6 ring atoms, and X is selected from —CH₂—, —CH(C₁-C₃alkyl)-, —C(C₁-C₃alkyl)₂-, —CH(cycloalkyl), —CH(NH₂)—, —C(CH₃)(NH₂)—, —CH(NH(C₁-C₃alkyl))-, —CH(N(C₁-C₃alkyl)₂)—, —CH(NH(cycloalkyl))-, —CH(NHCO(C₁-C₃alkyl))-, —CH(NHCO(cycloalkyl))-, —CH(NHSO₂(C₁-C₃alkyl))-, —CH(NHSO₂(cycloalkyl))-, —CH(OH)—, —CH(C₁-C₃alkoxy)-, —CH(cycloalkyloxy)-, —CO—, —SO₂—, —O—, —NH—, —N(C₁-C₃alkyl)-, —N(cycloalkyl)-, —CONH—, —CON(C₁-C₃alkyl)-, —CON(cycloalkyl)-, —N(CO(OC₁-C₃alkyl))-, —N(CO(O-cycloalkyl))-, —N(CO(CH₂OH))—, —SO₂NH—, —SO₂N(C₁-C₆alkyl)-, —SO₂N(cycloalkyl)-, —N(SO₂(C₁-C₃alkyl))-, —N(SO₂(cycloalkyl))-, —N(CO(C₁-C₃alkyl))-, or —N(CO(cycloalkyl))-.

5. A compound as claimed in claim 4 wherein X is —CH₂—, —SO₂—, —CO— (when adjacent to N), —O—, —N(SO₂(C₁-C₃alkyl))-, —N(SO₂(cycloalkyl))-, —N(CO(C₁-C₃alkyl))- —N(CO(cycloalkyl))-, —CONH—, —CON(C₁-C₃alkyl)-, or —CON(cycloalkyl)-.

6. A compound as claimed in claim 1 wherein R₁ and R₂, taken together with the carbon atom to which they are attached form an optionally substituted cyclopropyl, cyclobutyl, cyclopentyl, or cyclohexyl ring.

7. A compound as claimed in claim 6 wherein R₁ and R₂, taken together with the carbon atom to which they are attached form cyclopropyl ring.

8. A compound as claimed in claim 1 wherein R₁ and R₂, taken together with the carbon atom to which they are attached form a divalent ring of formula 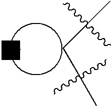
wherein the ring contains 4 to 6 ring atoms and X¹ is selected from —CH₂—, —CH(C₁-C₃alkyl)-, —C(C₁-C₃alky])₂—, —CH(cycloalkyl), —CH(NH₂)—, —CMe(NH₂)—, —CH(NH(C₁-C₃alkyl))-, —CH(N(C₁-C₃alkyl)₂)—, —CH(NH(cycloalkyl))-, —CH(NHCO(C₁-C₃alkyl))-, —CH(NHCO(cycloalkyl))-, —CH(NHSO₂(C₁-C₃alkyl))-, —CH(NHSO₂(cycloalkyl))-, —CH(OH)—, —CH(C₁-C₃alkoxy)-, —CH(cycloalkyloxy])-, —SO₂—, —O—, —NH—, —N(C₁-C₃alkyl)-, —N(cycloalkyl)-, —CONH—, —CON(C₁-C₃alkyl)-, —CON(cycloalkyl)-, —SO₂NH—, —SO₂N(C₁-C₆alkyl)-, —SO₂N(cycloalkyl)-, —N(SO₂(C₁-C₃alkyl))-, —N(SO₂(cycloalkyl))-, —N(CO(OC₁-C₃alkyl))-, —N(CO(O-cycloalkyl))-, —N(CO(CH₂OH))—, —N(CO(C₁-C₃alkyl))-, or —N(CO(cycloalkyl))-.

9. A compound as claimed in claim 8 wherein X¹ is —CH₂—, —SO₂—, —O—, —CONH—, —CON(C₁-C₃alkyl)-, —CON(cycloalkyl)-, —N(SO₂(C₁-C₃alkyl))-, —N(SO₂(cycloalkyl))-, —N(CO(C₁-C₃alkyl))-, or —N(CO(cycloalkyl))-.

10. A compound as claimed in claim 1 claims wherein A is hydrogen or methyl.

11. A compound as claimed in claim 1 wherein R is fluoro, chloro, bromo, (C₁-C₃)alkyl, cycloalkyl, trifluoromethyl, (C₁-C₃)alkoxy, (C₁-C₃)alkylmercapto, trifluoromethoxy, trifluoromethylthio, cyano, (C₁-C₃alkyl)SO₂—, NH₂SO₂—, (C₁-C₃alkyl)NHSO₂—, (C₁-C₃alkyl)₂NSO₂—, (cycloalkyl)NHSO₂—, NH₂CO—, (C₁-C₃alkyl)NHCO—, (C₁-C₃alkyl)₂NHCO—, or (cycloalkyl)NHCO—.

12. A compound as claimed in claim 1 wherein ring Ar is optionally substituted phenyl, pyridyl, pyrimidyl, diazolyl, oxazolyl, triazinyl, quinolinyl, pyrrollyl, furanyl, or thiazolyl.

13. A compound as claimed in claim 1 wherein optional substituents in Ar are selected from fluoro, chloro, bromo, (C₁-C₃)alkyl, cycloalkyl, trifluoromethyl, (C₁-C₃)alkoxy, trifluoromethoxy, trifluoromethylthio, cyano, NH₂CO—, (C₁-C₃alkyl)NHCO—, (C₁-C₃alkyl)₂NHCO—, (cycloalkyl)NHCO—, (C₁-C₃alkyl)SO₂—, NH₂SO₂—, (C₁-C₃alkyl)NHSO₂—, (cycloalkyl)NHSO₂—, and (C₁-C₃alkyl)₂NSO₂—.

14. A compound as claimed in claim 1 wherein Ar is a pyridone ring or a pyridine N-oxide ring.

15. A compound as claimed in claim 1 wherein any optional substituents in the phenyl ring containing R are selected from fluoro, chloro, bromo, cyano, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₃alkyl, fluoroC₁-C₂alkyl, difluoroC₁-C₂alkyl, C₁-C₃alkoxy, cycloalkyl, aryl, aryloxy, aryl(C₁-C₃)— or aryl(C₁-C₃alkoxy)-.

16. A pharmaceutical composition comprising a compound as claimed in claim 1, together with a pharmaceutically acceptable carrier.

17. (canceled)

18. A method of treatment of disease responsive to modulation of CRTH2 receptor activity comprising administering to a subject suffering such disease and effective amount of a compound as claimed in claim 1.

19. The method as claimed in claim 18, wherein the disease is one associated with elevated levels of prostaglandin D2 (PGD2) or one or more active metabolites thereof.

20. The method as claimed in claim 18, wherein the disease is an inflammatory, autoimmune, respiratory or allergy disease.

21. The method as claimed in claim 18, wherein the disease is selected from asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behçet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

22. The method as claimed in claim 18, wherein the disease is selected from asthma, rhinitis, allergic airway syndrome, and allergic rhinobronchitis.
